(19) United States
(12) Patent Application Publication
(10) Pub. No.: US 2026/0335109 A1
(43) Pub. Date: Oct. 29, 2026

(54) INTERNAL ACTUATION MECHANISM FOR A STENT DELIVERY SYSTEM

(71) Applicant: BOSTON SCIENTIFIC SCIMED, INC., Maple Grove, MN (US)

(72) Inventors: Peter L. Dayton, Brookline, MA (US); David Giles, Galway (IE); Fionn Stapleton, Oranmoe (IE); Keith O'Reilly, Kilcolgan (IE); Shane O'Keeffe, Tuam (IE); Daniel Tuck, Oughterard (IE); Gary Gilmartin, Foxford (IE); Mark Phillip Olson, St. Anthony, MN (US)

(73) Assignee: BOSTON SCIENTIFIC SCIMED, INC., Maple Grove, MN (US)

(21) Appl. No.: 19/660,698

(22) Filed: Apr. 28, 2026

Related U.S. Application Data
(60) Provisional application No. 63/796,865, filed on Apr. 29, 2025.

(51) Int. Cl. A61F 2/95; A61F 2/966; A61M 25/00; A61M 25/09

(52) U.S. Cl. CPC A61F 2/9517; A61F 2/966; A61M 2025/0004; A61M 25/0097; A61M 25/09

(57) ABSTRACT

This disclosure addresses deployment inaccuracies in conventional co-axial stent delivery systems. The disclosed stent delivery system includes a stationary proximal outer shaft, a retractable sleeve extending distal of the proximal outer shaft, and a moveable actuation shaft extending through a lumen of the proximal outer shaft and coupled to the retractable sleeve to deploy a self-expanding stent. By incorporating an internal actuation shaft rather than requiring retraction of the entire outer shaft, the systems reduces frictional deployment forces experienced by the stent delivery system.

### CROSS REFERENCE TO RELATED APPLICATIONS

The present application claims the benefit of U.S. Provisional Patent Application Serial No. 63/796,865, filed Apr. 29, 2025, the disclosure of which is incorporated herein by reference.

### TECHNICAL FIELD

The present disclosure generally relates to deployment of self-expanding stents in co-axial stent delivery systems. More specifically, the present disclosure is related to improving accuracy of stent placement and deployment by utilizing an internal actuation mechanism.

### BACKGROUND

In a conventional co-axial stent deployment system, a catheter shaft, which may be inserted through the working channel of an endoscope in some instances, may include an outer shaft slidably disposed over an inner shaft. To deploy a self-expanding stent therefrom, the outer shaft is withdrawn proximally relative to the inner shaft to expose the stent from the distal end of the outer shaft, thereby allowing the stent to radially expand from a radially constrained configuration within the outer shaft.

However, significant friction exists when attempting to withdraw the outer tube, such as between the outer shaft and the working channel of the endoscope, which may cause inadvertent movement of the catheter shaft, causing the stent to release prematurely and/or off-target. Thus, to reduce inaccuracies in stent deployment, a need to reduce and manage the frictional forces experienced by the stent delivery system, such as frictional forces between the stent delivery system and the working channel of an endoscope, is desired.

### BRIEF SUMMARY

In an example, a stent delivery system includes a handle assembly having a first actuatable handle and a second stationary handle, and a catheter shaft extending distally from the handle assembly. The catheter shaft includes a proximal outer shaft having a proximal end and distal end, where the proximal outer shaft includes a guidewire lumen and an actuation shaft lumen. The catheter shaft includes an inner shaft, where a proximal end region of the inner shaft is fixedly attached to the proximal outer shaft and extends distally therefrom, and where the inner shaft defines a guidewire lumen in communication with the guidewire lumen of the proximal outer shaft. A retractable sleeve surrounds the inner shaft, having a proximal sleeve region and a distal sleeve region. An actuation shaft extends distally through the actuation shaft lumen of the proximal outer shaft, where a proximal end of the actuation shaft is coupled to the first actuatable handle and a distal end of the actuation shaft is coupled to the retractable sleeve. A radially expandable stent surrounds a distal end region of the inner shaft. Actuation of the first actuatable handle relative to the second stationary handle retracts the retractable sleeve from a delivery configuration in which the distal sleeve region of the retractable sleeve surrounds and radially constrains the stent to a deployment configuration in which the stent is unconstrained by the retractable sleeve.

Alternatively or additionally to any of the examples above, the proximal sleeve region is configured to telescope proximally over the distal end of the proximal outer shaft when the retractable sleeve is retracted to the deployment configuration.

Alternatively or additionally to any of the examples above, a proximal end of the retractable sleeve surrounds the proximal outer shaft in both the delivery configuration and the deployment configuration.

Alternatively or additionally to any of the examples above, the system includes a collar positioned between the proximal sleeve region and the distal sleeve region, where the distal end of the actuation shaft is secured to the collar.

Alternatively or additionally to any of the examples above, the retractable sleeve surrounds the collar.

Alternatively or additionally to any of the examples above, the collar includes a tubular structure, having a proximal surface, a distal surface, and a collar lumen.

Alternatively or additionally to any of the examples above, the inner shaft extends through the collar lumen.

Alternatively or additionally to any of the examples above, the collar includes a tubular structure, having a proximal surface, a distal surface, a first collar lumen, and a second collar lumen.

Alternatively or additionally to any of the examples above, the actuation shaft extends into and/or through the first collar lumen and the inner shaft extends through the second collar lumen.

Alternatively or additionally to any of the examples above, the stent is positioned distal of the collar.

Alternatively or additionally to any of the examples above, the actuatable shaft extends along an exterior of the proximal end region of the inner shaft.

Alternatively or additionally to any of the examples above, the first actuatable handle is positioned proximal to the second stationary handle.

Alternatively or additionally to any of the examples above, the first actuatable handle is positioned distal to the second stationary handle.

Alternatively or additionally to any of the examples above, the proximal outer shaft includes a longitudinal channel and the actuation shaft is positioned in the channel.

Alternatively or additionally to any of the examples above, a proximal end region of the actuation shaft extends from the channel to couple with the first actuatable handle.

In an example, a stent delivery system includes a handle assembly having a first handle and a second handle, a proximal outer shaft fixed to and extending distally from the second handle, where the proximal outer shaft includes a guidewire lumen and an actuation shaft lumen. An inner shaft is fixedly attached to the proximal outer shaft and extends distally therefrom, where the inner shaft defines a guidewire lumen in communication with the guidewire lumen of the proximal outer shaft. A retractable sleeve has a distal sleeve region surrounding the inner shaft and defines a stent retention region therebetween. An actuation shaft is fixed to and extends distally from the first handle through the actuation shaft lumen of the proximal outer shaft and along an exterior of the inner shaft, where a distal end of the actuation shaft is coupled to the retractable sleeve. A radially expandable stent is disposed within the stent retention region. Actuation of the first handle relative to the second handle retracts the retractable sleeve from a delivery configuration in which the distal sleeve region of the retractable sleeve surrounds and radially constrains the stent to a deployment configuration in which the stent is unconstrained by the retractable sleeve.

Alternatively or additionally to any of the examples above, a proximal end of the retractable sleeve surrounds the proximal outer shaft in both the delivery configuration and the deployment configuration such that the retractable sleeve is configured to telescope proximally over the proximal outer shaft when the retractable sleeve is retracted to the deployment configuration.

In an example, a stent delivery system includes a handle assembly having a first actuatable handle and a second stationary handle, and a catheter shaft extending distally from the handle assembly. The catheter shaft includes a proximal outer shaft having a proximal end and distal end, where the proximal outer shaft includes a guidewire lumen and an actuation shaft lumen. The catheter shaft includes an inner shaft, where a proximal end region of the inner shaft is fixedly attached to the proximal outer shaft, such as a distal end region of the proximal outer shaft, and extends distally therefrom, and where the inner shaft defines a guidewire lumen in communication with the guidewire lumen of the proximal outer shaft. A retractable sleeve surrounds the inner shaft. The retractable sleeve includes a proximal sleeve region and a distal sleeve region. An actuation shaft extends distally through the actuation shaft lumen of the proximal outer shaft, where a proximal end of the actuation shaft is coupled to the first actuatable handle and a distal end of the actuation shaft is coupled to the retractable sleeve. A radially expandable stent surrounds a distal end region of the inner shaft. Actuation of the first actuatable handle relative to the second stationary handle retracts the retractable sleeve from a delivery configuration in which the distal sleeve region of the retractable sleeve surrounds and radially constrains the stent to a deployment configuration in which the stent is unconstrained by the retractable sleeve.

Alternatively or additionally to any of the example above, the retractable sleeve surrounds a distal end region of the proximal outer shaft.

Alternatively or additionally to any of the examples above, the proximal sleeve region is configured to telescope proximally over the distal end of the proximal outer shaft when the retractable sleeve is retracted to the deployment configuration.

Alternatively or additionally to any of the examples above, a proximal end of the retractable sleeve surrounds the proximal outer shaft in both the delivery configuration and the deployment configuration.

Alternatively or additionally to any of the examples above, the system includes a collar positioned between the proximal sleeve region and the distal sleeve region, where the distal end of the actuation shaft is secured to the collar.

Alternatively or additionally to any of the examples above, the retractable sleeve surrounds the collar.

Alternatively or additionally to any of the examples above, the collar includes a tubular structure, having a proximal surface, a distal surface, and a collar lumen.

Alternatively or additionally to any of the examples above, the inner shaft extends through the collar lumen.

Alternatively or additionally to any of the examples above, the collar includes a tubular structure, having a proximal surface, a distal surface, a first collar lumen, and a second collar lumen.

Alternatively or additionally to any of the examples above, the actuation shaft extends into and/or through the first collar lumen and the inner shaft extends through the second collar lumen.

Alternatively or additionally to any of the examples above, the stent is positioned distal of the collar.

Alternatively or additionally to any of the examples above, the actuatable shaft extends along an exterior of the proximal end region of the inner shaft.

Alternatively or additionally to any of the examples above, the first actuatable handle is positioned proximal to the second stationary handle.

Alternatively or additionally to any of the examples above, the first actuatable handle is positioned distal to the second stationary handle.

Alternatively or additionally to any of the examples above, the proximal outer shaft includes a longitudinal channel and the actuation shaft is positioned in the channel.

Alternatively or additionally to any of the examples above, a proximal end region of the actuation shaft extends from the channel to couple with the first actuatable handle.

In an example, a stent delivery system includes a handle assembly having a first handle and a second handle, a proximal outer shaft fixed to and extending distally from the second handle, where the proximal outer shaft includes a guidewire lumen and an actuation shaft lumen. An inner shaft is fixedly attached to the proximal outer shaft and extends distally therefrom, where the inner shaft defines a guidewire lumen in communication with the guidewire lumen of the proximal outer shaft. A retractable sleeve has a distal sleeve region surrounding the inner shaft and defines a stent retention region therebetween. An actuation shaft is fixed to and extends distally from the first handle through the actuation shaft lumen of the proximal outer shaft and along an exterior of the inner shaft, where a distal end of the actuation shaft is coupled to the retractable sleeve. A radially expandable stent is disposed within the stent retention region. Actuation of the first handle relative to the second handle retracts the retractable sleeve from a delivery configuration in which the distal sleeve region of the retractable sleeve surrounds and radially constrains the stent to a deployment configuration in which the stent is unconstrained by the retractable sleeve.

Alternatively or additionally to any of the examples above, a proximal end of the retractable sleeve surrounds the proximal outer shaft in both the delivery configuration and the deployment configuration such that the retractable sleeve is configured to telescope proximally over the proximal outer shaft when the retractable sleeve is retracted to the deployment configuration.

Alternatively or additionally to any of the examples above, the system includes a collar within the retractable sleeve between a proximal region of the retractable sleeve and a distal region of the retractable sleeve, where the distal end of the actuation shaft is secured to the collar.

Alternatively or additionally to any of the examples above, the inner shaft extends through a lumen of the collar.

In an example, a method of deploying a stent includes advancing a stent delivery system to a treatment site, where the stent delivery system includes a handle assembly having a first actuatable handle and a second stationary handle, and a catheter shaft extending distally from the handle assembly. The catheter shaft includes a proximal outer shaft having a proximal end and distal end, where the proximal outer shaft includes a guidewire lumen and an actuation shaft lumen. The catheter shaft includes an inner shaft, where a proximal end region of the inner shaft is fixedly attached to the proximal outer shaft and extends distally therefrom, and where the inner shaft defines a guidewire lumen in communication with the guidewire lumen of the proximal outer shaft. A retractable sleeve surrounds the inner shaft, having a proximal sleeve region and a distal sleeve region. An actuation shaft extends distally through the actuation shaft lumen of the proximal outer shaft, where a proximal end of the actuation shaft is coupled to the first actuatable handle and a distal end of the actuation shaft is coupled to the retractable sleeve. A radially expandable stent surrounds a distal end region of the inner shaft. The method includes actuating the first actuatable handle relative to the second stationary handle to retract the retractable sleeve from a delivery configuration in which the distal sleeve region surrounds and radially constrains the stent to a deployment configuration in which the stent is unconstrained by the retractable sleeve, where during retraction, the proximal sleeve region telescopes proximally over a distal end of the proximal outer shaft.

Alternatively or additionally to any of the examples above, the first actuatable handle is positioned proximal to the second stationary handle.

Alternatively or additionally to any of the examples above, the first actuatable handle is positioned distal to the second stationary handle.

### BRIEF DESCRIPTION OF THE SEVERAL VIEWS OF THE DRAWINGS

The disclosure may be more completely understood in consideration of the following detailed description in connection with the accompanying drawings, in which:

FIG. 1 is a partial cross-sectional side view of an exemplary stent delivery system.

FIG. 2 is a cross-sectional view of a portion of the exemplary stent delivery system of FIG. 1 taken along line 2-2.

FIG. 3 is a cross-sectional view of a portion of the exemplary stent delivery system of FIG. 1 taken along line 3-3.

FIG. 4 is a partial cross-sectional side view of the exemplary stent delivery system of FIG. 1 after deployment of the stent.

FIG. 5 illustrates a portion of a stent delivery system in accordance with another embodiment of the subject matter disclosed herein.

FIG. 5A is a cross-sectional view of a portion of the exemplary stent delivery system of FIG. 5 taken along line 5A-5A.

FIG. 6 illustrates the portion of the stent delivery system of FIG. 5 after deployment of the stent.

FIG. 6A is a cross-sectional view of a portion of the exemplary stent delivery system of FIG. 6 taken along line 6A-6A.

FIG. 7 is a perspective view of a portion of an exemplary stent delivery system.

FIG. 8 is a perspective view of a portion of an alternative construction of the exemplary stent delivery system.

FIG. 8A is a cross-sectional view along line 8-8 of FIG. 8.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

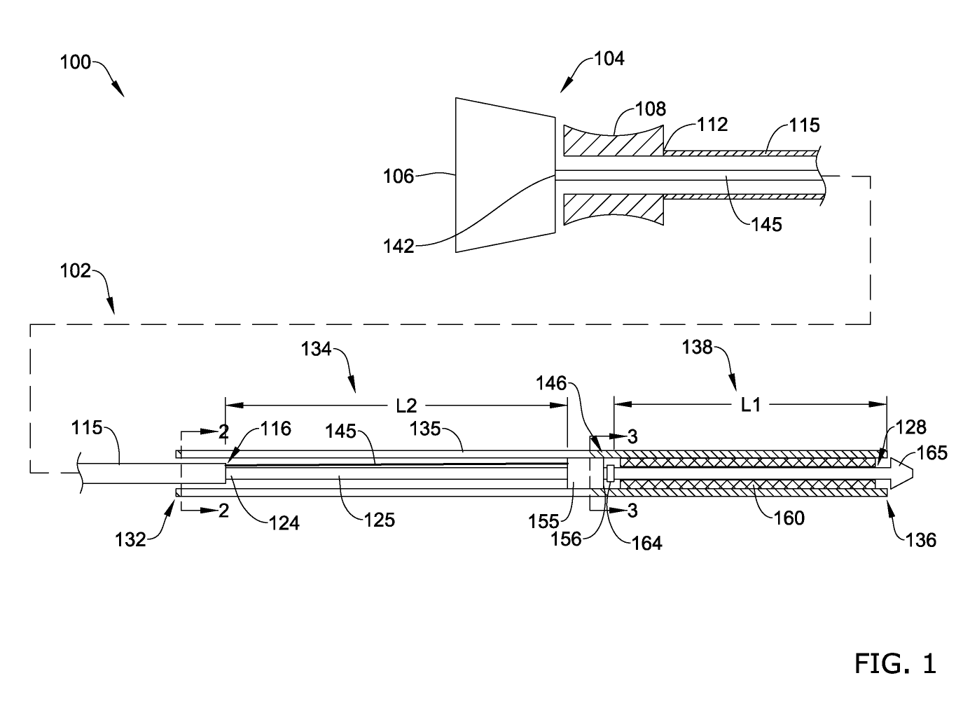

FIG. 1 shows a side view of a stent delivery system 100 with some components shown in cross-section. Stent delivery system 100 includes a handle assembly 104 and a catheter shaft 102 extending distally from the handle assembly 104, to deliver and deploy a radially expandable stent 160 in a body lumen of a patient. The catheter shaft 102 may include a proximal outer shaft 115, an inner shaft 125, a retractable sleeve 135, an actuation shaft 145, and a collar 155.

To operate the stent delivery system 100, the handle assembly 104 includes a first, actuatable handle 106 and a second, stationary handle 108. The first, actuatable handle 106 may be actuated relative to the second stationary handle 108 to release the stent 160 from a delivery configuration to a deployment configuration. In the embodiment illustrated in FIG. 1 the first, actuatable handle 106 is positioned proximal to the second, stationary handle 108.

The proximal outer shaft 115 is fixed in position and secured to the second, stationary handle 108, such as fixed within or at an end of the second, stationary handle 108 of the handle assembly 104. The proximal outer shaft 115 may be secured to the second, stationary handle 108 and extend distally therefrom. Additionally, a proximal end region 124 of the inner shaft 125 is fixedly attached or secured to the proximal outer shaft 115, such as secured to a distal end region of the proximal outer shaft 115. The inner shaft 125 extends distally from the distal end of the proximal outer shaft 115 to a distal tip 165 fixed at a distal end of the inner shaft 125.

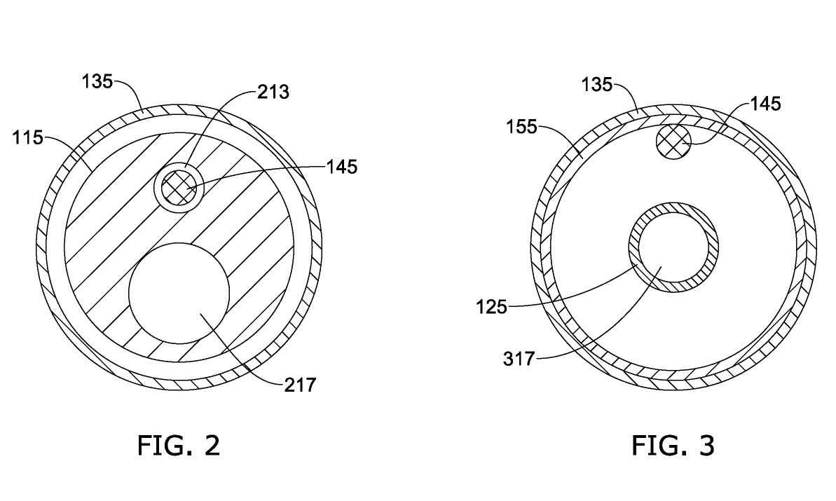

Briefly referring to FIG. 2, it can be seen in the cross-sectional illustration that the proximal outer shaft 115 may include an actuation shaft lumen 213 and a guidewire lumen 217. In some instances, the proximal outer shaft 115 may be a multi-lumen tubular member formed of a monolithic construction defining both the actuation shaft lumen 213 and the guidewire lumen 217. Other shaft configurations are also contemplated. Although the proximal outer shaft 115 is illustrated with two lumens, additional lumens may be present.

FIG. 1 illustrates an over-the-wire stent delivery system, wherein the guidewire lumen 217 extends longitudinally from the proximal end of the proximal outer shaft to the distal end of distal tip 165. In this type of stent delivery system 100, the user may initially place the guidewire in the patient, then slide the catheter shaft 102 over the guidewire to place the catheter shaft 102, with the stent 160 loaded therein, at the target site. However, in some embodiments (not illustrated) the stent delivery system 100 may be configured as a rapid exchange stent delivery system in which the proximal outer shaft 115 includes a guidewire port located distal of the proximal end of the proximal outer shaft 115 that is in communication with the guidewire lumen 217 to allow a guidewire to exit the guidewire lumen 217 and extend along an exterior of the proximal outer shaft 115 proximal of the guidewire port. In some instances the guidewire port may be located between 30 centimeters (cm) and 60 cm from the distal tip 165, or about 40 cm from the distal tip 165.

A proximal end 142 of the actuation shaft 145 may be fixedly coupled or secured to the first, actuatable handle 106 and extend distally therefrom. For example, the actuation shaft 145 may be secured to the first, actuatable handle 106 and extend distally therefrom through the actuation shaft lumen 213.

The inner shaft 125 may be fixed to the guidewire lumen 217 of the proximal outer shaft 115 such that the lumen of the inner shaft 125 is in communication with the guidewire lumen 217 of the proximal outer shaft 115 such tht the lumen of the inner shaft 125 may define an extension of the guidewire lumen extending through the inner shaft 125 to the distal tip 165.

Referring back to FIG. 1, the retractable sleeve 135, which may be a tubular member, is illustrated in a delivery configuration (i.e., prior to stent deployment). The retractable sleeve 135 is configured to surround the inner shaft 125 and retain the stent 160 is a radially contracted, delivery configuration. When the retractable sleeve 135 is in the delivery configuration, the stent 160 surrounds a distal end region 128 of the inner shaft 125 and is radially constrained by the distal sleeve region 138 of the retractable sleeve 135.

Among other considerations later discussed, the length of the retractable sleeve 135 may be chosen to ensure that the distal end 136 of the retractable sleeve 135 always remains in a working channel of an endoscope throughout operation of the stent delivery system 100 in order to prevent interference with the elevator of the endoscope.

The retractable sleeve 135 includes two regions, the distal sleeve region 138 and a proximal sleeve region 134 extending proximal of the distal sleeve region 138, both surrounding the inner shaft 125. In other words, the inner shaft 125 may extend through the lumen of the retractable sleeve 135 throughout both the proximal sleeve region 134 and the distal sleeve region 138. A collar 155 may be secured or otherwise fixed to the retractable sleeve 135 between the proximal sleeve region 134 and the distal sleeve region 138.

The distal sleeve region 138 may define a stent encapsulation pocket along length L1 of the inner shaft 125, where the stent 160 is radially constrained between an inner surface of the distal sleeve region 138 and an outer surface of the inner shaft 125. Length L1 has a length sufficient to accommodate the full length of the stent 160 in its radially constrained configuration during delivery of the stent 160.

In some embodiments, the stent delivery system 100 may include a stent retention bumper or retainer 164, or a plurality of stent retention bumpers or retainers 164. In some examples, length L1 of the stent encapsulation pocket may be defined as the length between the stent retention bumper or retainer 164 and the distal tip 165. The stent retention bumper or retainer 164 may be any desired shape configured to engage a portion of the stent 160 when radially constrained. In some instances, the stent retention bumper or retainer 164 may be a tubular structure surrounding the inner shaft 125 and having a larger outer diameter than the outer diameter of the inner shaft 125 extending therebetween, however other means of coupling are contemplated. In some embodiments (not illustrated), the stent delivery system 100 may include two stent retention bumpers, a distal stent retention bumper located proximate to the distal end of the stent 160 and a proximal stent retention bumper located proximate to the proximal end of the stent 160, to define the length L1 of the stent encapsulation pocket therebetween.

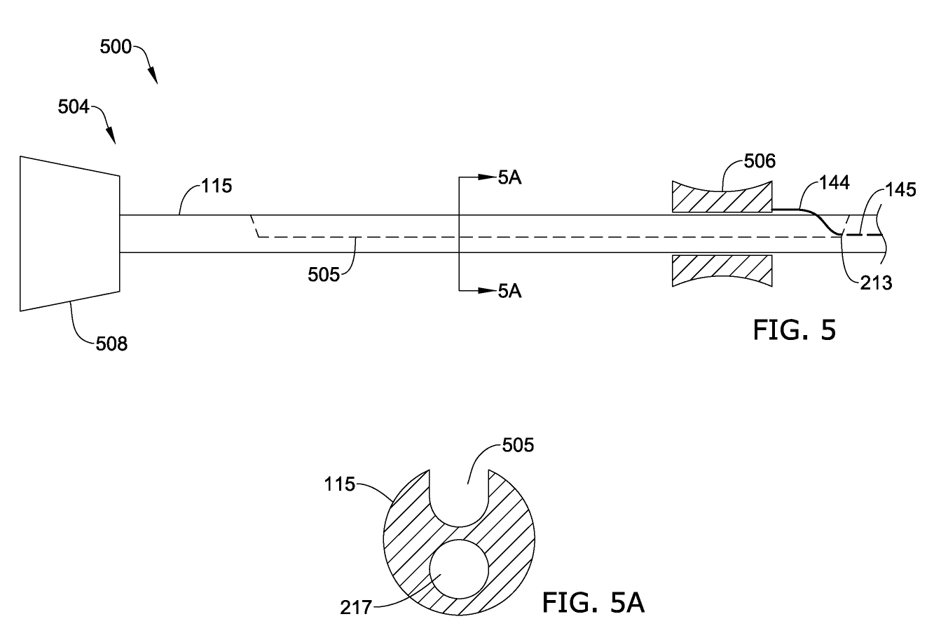

The stent retention bumper(s) or retainer(s) 164 may be configured to keep the longitudinal position of the stent 160 relative to the inner shaft 125 steady, preventing axial movement the stent 160 from following the movement of the retractable sleeve 135 as the retractable sleeve 135 is withdrawn proximally as the retractable sleeve 135 transitions into a deployment configuration for releasing the stent 160 (see FIG. 4). Including the stent retention bumper(s) or retainer(s) 164 prevents the stent 160 from following or responding to movements of the handle assembly 104 and/or the catheter shaft 102 prior to full deployment, further ensuring that the stent 160 is deployed on-target. In embodiments that include stent retention bumpers or retainers 164, the distal end 136 of the retractable sleeve 135 may be located distal to the stent retention bumpers or retainers 164 in the delivery configuration.

The distance between the distal end 116 of the proximal outer shaft 115 and the proximal end of the collar 155 has a length L2 in the delivery configuration in which the stent 160 is radially constrained within the distal sleeve region 138. Length L2 has a sufficient length for the proximal sleeve region 134 to telescope over the distal end 116 of the proximal outer shaft 115 as the retractable sleeve 135 is withdrawn proximally and transitions to the deployment configuration. For instance, a proximal end region of the retractable sleeve 135 may surround the proximal outer shaft 115 (e.g., a distal end region of the outer shaft 115) in both the delivery configuration (FIG. 1) and the deployment configuration (FIG. 4) and the length L2 may be greater than the length of the stent 160 in its radially constrained configuration when constrained within the distal sleeve region 138 in the delivery configuration during delivery to provide sufficient travel, for collar 155 to move proximally toward the distal end 116 of the proximal outer shaft 115 as the proximal sleeve region 134 telescopes proximally over the proximal outer shaft 115 during stent deployment to fully uncover the stent 160. The length L2 of the proximal sleeve region 134 may also be greater than the length L1 of the distal sleeve region 138 to provide a margin of safety that ensures the constrained stent 160 will be fully uncovered when the proximal sleeve region 134 telescopes proximally. Additionally, a greater length L2 relative to L1 may provide assurance that the proximal end 132 of the retractable sleeve 135 will remain within the working channel of the endoscope throughout the delivery and deployment of the stent 160. In some instances, the length L1 may be 5 centimeters (cm) or more, 6 cm or more, 7 cm or more, 8 cm or more, 9 cm or more, or 10 cm or more. In some instances, the length L2 may be 20 cm or more, 25 cm or more, 28 cm or more, or 30 cm or more. In some instances, the length L2 may be in the range of 20 cm to 50 cm, in the range of 20 cm to 40, or in the range of 20 cm to 30 cm, for example.

In the illustrated example, the proximal sleeve region 134 may surround, i.e., axially overlap, with a distal end region of the proximal outer shaft 115. For instance, referring again to FIG. 2, the retractable sleeve 135 is shown having an inner diameter greater than an outer diameter of the proximal outer shaft 115 with the retractable sleeve 135 surrounding the proximal outer shaft 115. In other words, the proximal end 132 of the retractable sleeve 135 may be positioned proximal of the distal end 116 of the proximal outer shaft 115 in both the delivery configuration (FIG. 1) and the deployment configuration (FIG. 4). The extent of acceptable axial overlap may be affected by various factors, including but not limited to, containing the proximal end 132 of the retractable sleeve 135 within the working channel of the endoscope throughout the stent delivery procedure and ensuring that the proximal end 132 of the retractable sleeve 135 surrounds the proximal outer shaft 115 and is axially slidable relative thereto, regardless of whether the retractable sleeve 135 is in the delivery configuration or the deployment configuration. The proximal end 132 of the retractable sleeve 135 may be spaced distally from the handle assembly 104 and unattached thereto, with a length of the proximal outer shaft 115 extending proximally from the proximal end 132 of the retractable sleeve 135 to the second, stationary handle 108 of the handle assembly 104. The length of the proximal outer shaft 115 may be greater than the length of the retractable sleeve 135, and in some instances, the length of the proximal outer shaft 115 may be significantly greater than the length of the retractable sleeve 135, e.g., 2 or more times greater, 3 or more times greater, or 4 or more times greater, in some instances.

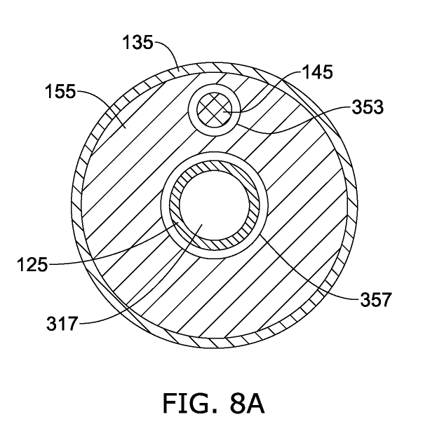

The distal end 146 of the actuation shaft 145 may be secured or fixed to a medial region of the retractable sleeve 135 with the proximal sleeve region 134 extending proximal of the medial region and the distal sleeve region 138 extending distal of the medial region. For example, the collar 155 may be positioned within the medial region of the retractable sleeve 135 and the distal end 146 of the actuation shaft 145 secured or fixed thereto. In some instances, the collar 155, such as a tubular collar, may be secured within the lumen of the retractable sleeve 135 with the distal end 146 of the actuation shaft 145 secured to or fixed to (e.g., welded, bonded, etc.) to the collar 155. In some embodiments, the distal end 146 of the actuation shaft 145 may extend through the collar 15 and be positioned distal to the distal end 156 of collar 155 (see also FIG. 8) to secure the distal end 146 of the actuation shaft 145 to the collar 155.

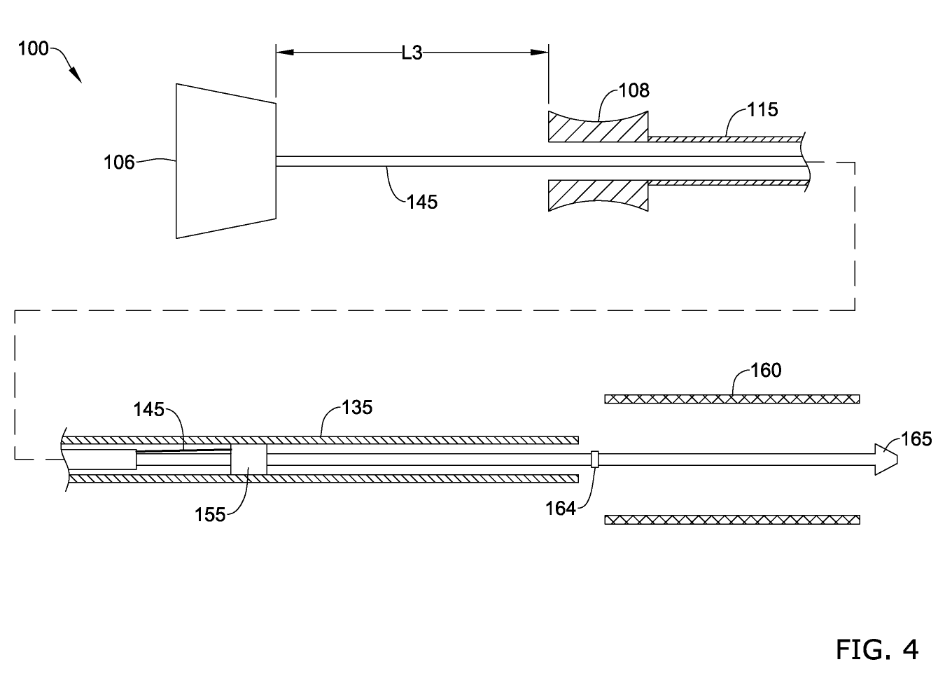

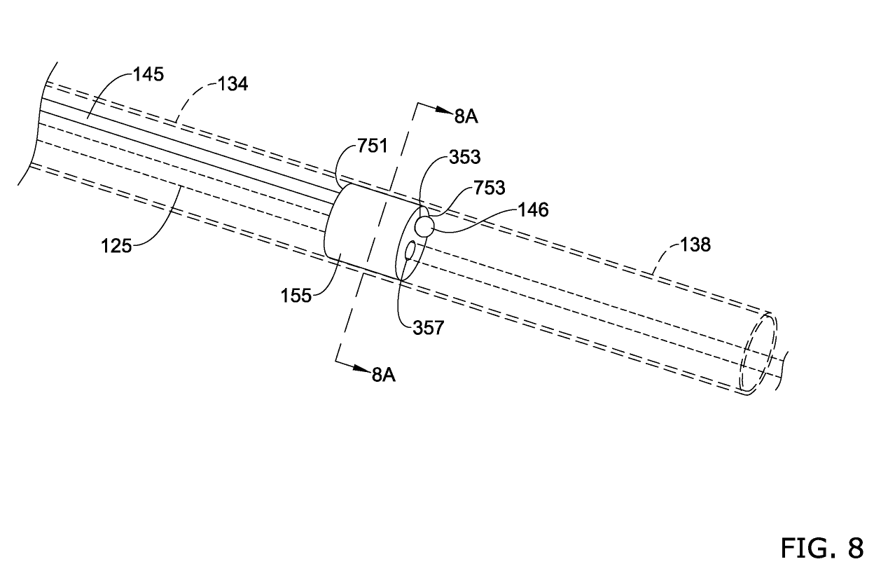

FIG. 3 shows a cross-sectional view of the catheter shaft 102, showing the collar 155 located between the distal sleeve region 138 and the proximal sleeve region 134. The collar 155 may serve as the connection point between the actuation shaft 145 and the retractable sleeve 135. In some examples, the collar 155 may be secured to the retractable sleeve 135 with a close fit, with the retractable sleeve 135 surrounding the collar 155. In some instances, the collar 155 may be bonded to and/or form an interference fit with an inner surface of the retractable sleeve 135. A close fit may ensure proper transfer of the actuation motion applied to the actuation shaft 145 to the retractable sleeve 135 and prevent inadvertent or unwanted movement between the components of the stent delivery system 100 which could affect the precision of deployment of stent 160. A distal end 146 of the actuation shaft 145 may be fixedly attached to the collar 155, thus securing the distal end 146 of the actuation shaft 145 with the retractable sleeve 135. In some embodiments, the distal end 146 of the actuation shaft 145 may be secured to a proximal end of the collar 155, may be secured within the lumen of the collar 155, or may extend distally beyond the collar 155 to be secured to a distal end of the collar (see FIG. 7). The actuation shaft 145 may extend proximally from the collar 155 along an exterior of the inner shaft 125 and into the actuation shaft lumen 213 of the proximal outer shaft 115. The actuation shaft 145 may extend through the actuation shaft lumen 213 of the proximal outer shaft 115 to the actuatable handle 106 of the handle assembly 104.

The inner shaft 125 may extend through the lumen of the collar 155 such that the collar 155 is longitudinally movable relative to the inner shaft 125. In some embodiments, the inner shaft 125 defines a guidewire lumen 317 extending through the collar 155. The defined guidewire lumen 317 is in communication with the guidewire lumen 217 of the proximal outer shaft 115 to form a continuous pathway for guidewire passage from the proximal outer shaft 115 through the inner shaft 125 to the distal tip 165. The guidewire passage may be used to provide a path for the advancement of the stent delivery system 100 over a guidewire to position the stent 160, and otherwise help in accurate deployment.

FIG. 4 illustrates a side view of the stent delivery system 100 when the retractable sleeve 135 is in the deployment configuration with some components shown in cross-section. More specifically, FIG. 4 illustrates an embodiment of the stent delivery system 100 in which the handle assembly 104 is configured with the first, actuatable handle 106 is positioned proximal to the second, stationary handle 108.

Once the stent 160 has been advanced within the catheter shaft 102 to a target site within the body lumen of the patient, the user may actuate (e.g., move proximally) the first, actuatable handle 106 relative to the second, stationary handle 108 such that the first, actuatable handle 106 moves away from the second, stationary handle 108, for a length L3. Length L3 must be at least of sufficient length to uncover the full axial length of the constrained stent 160. Length L3 is sufficient if, when the first, actuatable handle 106 is moved proximally, the actuation shaft 145 is also retracted proximally by the same length L3. As a result of retracting the actuation shaft 145 by a length of L3, the retractable sleeve 135, coupled to the actuation shaft 145 via the collar 155, also moved proximally by the same length L3 relative to stent 160, in turn exposing and releasing the stent 160 from its radially constrained configuration to a radially expanded, deployed configuration placed at the target site. In sum, actuating the first, actuatable handle 106 relative to the second, stationary handle 108 causes the actuation shaft 145, the collar 155, and the retractable sleeve 135 to proximally move as a unit while the proximal outer shaft 115 and the inner shaft 125 remain stationary to uncover stent 160.

The stent retention bumpers or retainers 164 mounted on the inner shaft 125 remain immobile as the retractable sleeve 135 retracts proximally into the deployment configuration, acting as a stop for the stent 160 as the retractable sleeve 135 is pulled proximally relative to the proximal outer shaft 115 and the inner shaft 125, such that the stent 160 does not follow the movement of the retractable sleeve 135 and is deployed on-target.

In conventional stent delivery systems, a singular outer shaft is secured to a first, actuatable handle and extends distally up to a distal tip of the delivery system to compressably constrain a stent within the outer shaft proximal to the distal tip. In the conventional system, stent deployment is achieved by proximal retraction of the outer shaft relative to an inner shaft that the radially constrained stent surrounds, with the inner shaft secured to a second, stationary handle. However, in stent delivery system 100, because the actuation shaft 145 is internal to the proximal outer shaft 115 and axially moveable relative thereto, the proximal outer shaft 115 may remain stationary. This allows the user, typically a physician or medical technician, to hold onto the stationary components of the system 100, such as the proximal outer shaft 115 or the second, stationary handle 108 (to which the proximal outer shaft 115 is fixedly attached) during operation without disturbance to the system's actuation mechanism and consequently preserving the positioning of the stent 160.

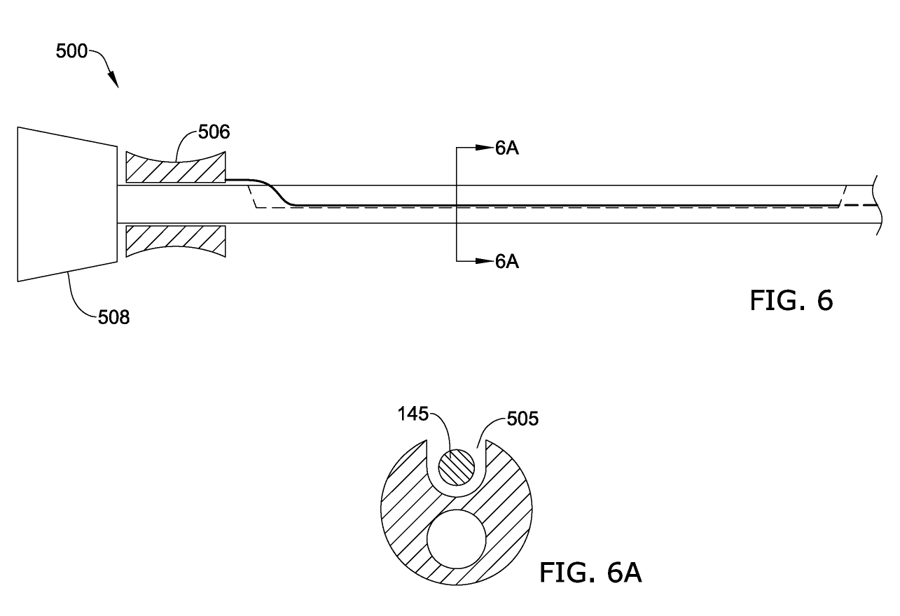

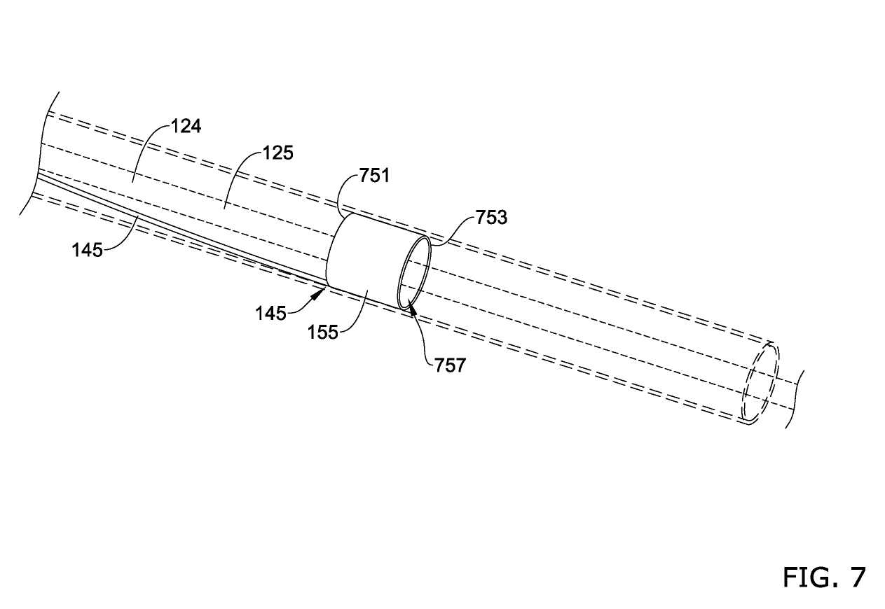

FIGS. 5 and 6 show a stent delivery system 500 with an alternative handle assembly 504 configuration, wherein the first, actuatable handle 506, which is actuatable, is positioned distal to the second, stationary handle 508. FIG. 5 shows a side view of the handle assembly 504 of the stent delivery system 500 when the retractable sleeve 135 is in the delivery configuration as shown in FIG. 1, whereas FIG. 6 shows a side view of the handle assembly 504 of the stent delivery system 500 when the retractable sleeve 135 is in the deployment configuration as shown in FIG. 4. The proximal end of the actuation shaft 145 may be fixed to the first, actuatable handle 506 and extend distally therefrom. Likewise, the proximal end of the proximal outer shaft 115 may be fixed to the second, stationary handle 508 and extend distally therefrom.

In this embodiment, the proximal outer shaft 115 may include a longitudinal channel 505, within which the actuation shaft 145 may be positioned. The longitudinal channel 505 may run along a portion of the length of the proximal outer shaft 115 to serve as a pathway for the proximal end region 144 of the actuation shaft 145 to travel, allowing the proximal end region 144 of actuation shaft 145 to exit the actuation shaft lumen 213 of the proximal outer shaft 115, travel along the exterior of the proximal outer shaft 115, and couple to the first, actuatable handle 506 for operation. Additionally, the longitudinal channel 505 helps keep the actuation shaft 145 properly guided and contained, preventing inadvertent movement or interference that could lead to inaccuracies during deployment.

FIG. 5A shows a cross-sectional view of the proximal outer shaft 115 taken along line 5A-5A of FIG. 5. The proximal outer shaft 115 includes the guidewire lumen 217 and the longitudinal channel 505, which is in communication with the actuation shaft lumen 213 extending distally of the longitudinal channel 505 through the proximal outer shaft 115 to the distal end of the outer shaft 115. In this example, the longitudinal channel 505 is illustrated with a U-shaped cross-section opening laterally to a periphery of the proximal outer shaft 115. In some examples, the cross-sectional shape may be specifically selected to reflect the cross-sectional shape of the actuation shaft 145. In this example, a U-shaped cross-section may be selected for an actuation shaft 145 that is a rounded pull wire. However, a match in the cross-sectional shapes of the longitudinal channel 505 and the actuation shaft 145 is not required.

To deploy the stent 160, the user may actuate the first, actuatable handle 506 (i.e., move proximally) relative to the second, stationary handle 508 such that the first, actuatable handle 506 moves towards the second, stationary handle 508. Actuation of the first, actuatable handle 506 actuates the actuation shaft 145 proximally relative to the proximal outer shaft 115, which in turn withdraws the retractable sleeve 135 proximally over and relative to the distal end of the proximal outer shaft 115 into the delivery configuration, resulting in deployment of the stent 160 in a similar manner to the stent delivery system 100 of FIG. 4.

The longitudinal channel 505 may have a length sufficient to accommodate the full length of movement required by the actuation shaft 145 during deployment. In the illustrated example, the length of the longitudinal channel 505 is such that the first, actuatable handle 506 and the second, stationary handle 508 abut against each other once in the deployment configuration. However, in other examples, the first, actuatable handle 506 and the second, stationary handle 508 may not abut in the deployment configuration because the retractable sleeve 135 has already traveled a sufficient length to release the stent 160 from its radially constrained configuration.

FIG. 6A shows a cross-sectional view of the proximal outer shaft 115 at line 6A-6A of FIG. 6, showing the actuation shaft 145 positioned in the longitudinal channel 505. In this example, the actuation shaft 145 is a rounded pull wire, however the actuation shaft 145 may differ in shape so long as it is able to move through the track created by the longitudinal channel 505.

FIG. 7 is a partial perspective view of a portion of the stent delivery system 100 centered at the collar 155 and the surrounding regions 134, 138 of the retractable sleeve 135 extending in opposite directions from the collar 155. In some examples, the collar 155 is a tubular structure with a proximal surface 751 at a proximal end of the collar 155 and a distal surface 753 at a distal end of the collar 155. The tubular structure of the collar 155 may define a collar lumen 757 extending therethrough from the proximal end or proximal surface 751 to the distal end or distal surface 753 of the collar 155. The lumen 757 of the collar 155 may serve as a structural opening for which the inner shaft 125 may pass through the collar 155. The lumen 757 of the collar 155 may be concentric with the tubular wall of the collar 155 to provide consistent spacing between the inner shaft 125 and the retractable sleeve 135 in all directions.

In some embodiments, the distal end 146 of the actuation shaft 145 may be eccentrically attached to the collar 155, such that the securement point between the distal end of the actuation shaft 145 and the collar 155 is not concentric with the lumen 757 extending through the collar 155. In some instances, the distal end of the actuation shaft 145 may be affixed or secured to the proximal surface 751 of the collar 155 and/or an inner surface of the collar 155 offset from the central longitudinal axis of the collar 155. In the illustrated example, the distal end 146 of the actuation shaft 145 is attached to the proximal surface 751 of the collar 155 at its outermost edge. However, any eccentric positioning which creates the necessary clearance for the inner shaft 125 to pass through the lumen 757 of the collar 155 may be acceptable. This eccentric arrangement enables the actuation shaft 145 to extend along the exterior of the proximal end region 124 of the inner shaft 125 with the inner shaft 125 extending through the lumen 757 of the collar 155 and distally thereto.

FIGS. 8 and 8A illustrate an alternative configuration of the collar 155 and securement of the actuation shaft 145 thereto. As shown in the embodiment of FIGS. 8 and 8A, the collar 155 may include two lumens, a first collar lumen 353 and a second collar lumen 357. A distal end 146 of the actuation shaft 145 may be fixedly attached to the first collar lumen 353, thus securing the distal end 146 of the actuation shaft 145 with the retractable sleeve 135. For example, the actuation shaft 145 may extend into and/or through the first collar lumen 353 with the distal end 146 of the actuation shaft 145 fixed thereto. The actuation shaft 145 may extend proximally from the collar 155 along an exterior of the inner shaft 125 and into the actuation shaft lumen 213 of the proximal outer shaft 115. The actuation shaft 145 may extend through the actuation shaft lumen 213 of the proximal outer shaft 115 to the actuatable handle 106 of the handle assembly 104.

As illustrated in FIG. 8, the actuation shaft 145 may pass through the first collar lumen 353 from the proximal surface 751 to the distal surface 753 of the collar 155. In this embodiment, the distal end 146 of the actuation shaft 145 may be an enlarged protuberance (e.g., a welded bead, a swaged end, etc.) with a greater diameter than the first collar lumen 353 so that the distal end 156 of the actuation shaft 145 is prevented from passing proximally through the first collar lumen 353 during deployment of the stent 160. The distal end 146 of the actuation shaft 145 is not limited to a welded bead as illustrated, rather the distal end 146 may take other shapes in other embodiments, precluding the distal end 146 of the actuation shaft 145 from passing proximally through the first collar lumen 353.

In other embodiments (not illustrated), the actuation shaft 145, such as the distal end 146, may be fixedly attached at other points within the first collar lumen 353 or to the proximal surface 751 of the collar 155. These embodiments may also provide a securement point between the distal end 146 of the actuation shaft 145 and the collar 155, such that the actuation shaft 145 is coupled to the retractable sleeve 135 by way of the coupling between the collar 155 and the retractable sleeve 135 (see FIG. 3).

In some embodiments, the actuation shaft 145 may have a reduced diameter from the portion extending between the proximal surface 751 of the collar 155 and the distal surface 753 of the collar 155 relative to a diameter of the actuation shaft 145 extending proximally from the proximal surface 751 of the collar 155. Accordingly, the reduced diameter of the actuation shaft 145 may form a shoulder at the proximal surface 751, and thus the shoulder may abut the proximal surface 751. The shoulder of the actuation shaft 145 may prevent distal movement of the actuation shaft 145 relative to the collar 155, thus enabling distal actuation of the collar 155, and thus distal actuation of the retractable sleeve 135 via distal movement of the actuation shaft 145.

The inner shaft 125 may extend through the second collar lumen 357 of the collar 155. The inner shaft 125 may be non-affixed to the second collar lumen 357 such tht the collar 155 is longitudinally movable relative to the inner shaft 125. In some embodiments, the inner shaft 125 defines a guidewire lumen 317 extending through the collar 155, as described above.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent delivery system comprising:
a handle assembly including a first actuatable handle and a second stationary handle;
a catheter shaft extending distally from the handle assembly, the catheter shaft comprising:
a proximal outer shaft, having a proximal end and distal end,
wherein the proximal outer shaft includes a guidewire lumen and an actuation shaft lumen,
an inner shaft,
wherein a proximal end region of the inner shaft is fixedly attached to the proximal outer shaft and extends distally therefrom, and
wherein the inner shaft defines a guidewire lumen in communication with the guidewire lumen of the proximal outer shaft;
a retractable sleeve surrounding the inner shaft, the retractable sleeve having a proximal sleeve region and a distal sleeve region; and
an actuation shaft extending distally through the actuation shaft lumen of the proximal outer shaft;
wherein a proximal end of the actuation shaft is coupled to the first actuatable handle and a distal end of the actuation shaft is coupled to the retractable sleeve, and
a radially expandable stent surrounding a distal end region of the inner shaft;
wherein actuation of the first actuatable handle relative to the second stationary handle retracts the retractable sleeve from a delivery configuration in which the distal sleeve region of the retractable sleeve surrounds and radially constrains the stent to a deployment configuration in which the stent is unconstrained by the retractable sleeve.

2. The stent delivery system of claim 1, wherein the proximal sleeve region is configured to telescope proximally over the distal end of the proximal outer shaft when the retractable sleeve is retracted to the deployment configuration.

3. The stent delivery system of claim 1, wherein a proximal end of the retractable sleeve surrounds the proximal outer shaft in both the delivery configuration and the deployment configuration.

4. The stent delivery system of claim 1, further comprising a collar positioned between the proximal sleeve region and the distal sleeve region, the distal end of the actuation shaft secured to the collar.

5. The stent delivery system of claim 4, wherein the retractable sleeve surrounds the collar.

6. The stent delivery system of claim 4, wherein the collar comprises a tubular structure, having a proximal surface, a distal surface, a first collar lumen and a second collar lumen.

7. The stent delivery system of claim 6, wherein the actuation shaft extends into the first collar lumen and the inner shaft extends through the second collar lumen.

8. The stent delivery system of claim 4, wherein the stent is positioned distal of the collar.

9. The stent delivery system of claim 1, wherein the actuatable shaft extends along an exterior of the proximal end region of the inner shaft.

10. The stent delivery system of claim 1, wherein the first actuatable handle is positioned proximal to the second stationary handle.

11. The stent delivery system of claim 1, wherein the first actuatable handle is positioned distal to the second stationary handle.

12. The stent delivery system of claim 11, wherein the proximal outer shaft includes a longitudinal channel and the actuation shaft is positioned in the channel.

13. The stent delivery system of claim 12, wherein a proximal end region of the actuation shaft extends from the channel to couple with the first actuatable handle.

14. A stent delivery system comprising:
a handle assembly including a first handle and a second handle;
a proximal outer shaft fixed to and extending distally from the second handle, the proximal outer shaft including a guidewire lumen and an actuation shaft lumen;
an inner shaft fixedly attached to the proximal outer shaft and extending distally therefrom, the inner shaft defining a guidewire lumen in communication with the guidewire lumen of the proximal outer shaft;
a retractable sleeve having a distal sleeve region surrounding the inner shaft and defining a stent retention region therebetween; and
an actuation shaft fixed to and extending distally from the first handle through the actuation shaft lumen of the proximal outer shaft and along an exterior of the inner shaft, a distal end of the actuation shaft being coupled to the retractable sleeve; and
a radially expandable stent disposed within the stent retention region;
wherein actuation of the first handle relative to the second handle retracts the retractable sleeve from a delivery configuration in which the distal sleeve region of the retractable sleeve surrounds and radially constrains the stent to a deployment configuration in which the stent is unconstrained by the retractable sleeve.

15. The stent delivery system of claim 14, wherein a proximal end of the retractable sleeve surrounds the proximal outer shaft in both the delivery configuration and the deployment configuration such that the retractable sleeve is configured to telescope proximally over the proximal outer shaft when the retractable sleeve is retracted to the deployment configuration.

16. The stent delivery system of claim 14, further comprising a collar within the retractable sleeve between a proximal region of the retractable sleeve and a distal region of the retractable sleeve, the distal end of the actuation shaft secured to the collar.

17. The stent delivery system of claim 16, wherein the inner shaft extends through a lumen of the collar.

18. A method of deploying a stent comprising:
advancing a stent delivery system to a treatment site, the stent delivery system comprising:
a handle assembly including a first actuatable handle and a second stationary handle;
a catheter shaft extending distally from the handle assembly, the catheter shaft comprising:
a proximal outer shaft, having a proximal end and distal end,
wherein the proximal outer shaft includes a guidewire lumen and an actuation shaft lumen,
an inner shaft,
wherein a proximal end region of the inner shaft is fixedly attached to the proximal outer shaft and extends distally therefrom, and
wherein the inner shaft defines a guidewire lumen in communication with the guidewire lumen of the proximal outer shaft;
a retractable sleeve surrounding the inner shaft, the retractable sleeve having a proximal sleeve region and a distal sleeve region; and
an actuation shaft extending distally through the actuation shaft lumen of the proximal outer shaft;
wherein a proximal end of the actuation shaft is coupled to the first actuatable handle and a distal end of the actuation shaft is coupled to the retractable sleeve, and
a radially expandable stent surrounding a distal end region of the inner shaft;
actuating the first actuatable handle relative to the second stationary handle to retract the retractable sleeve from a delivery configuration in which the distal sleeve region surrounds and radially constrains the stent to a deployment configuration in which the stent is unconstrained by the retractable sleeve, wherein during retraction, the proximal sleeve region telescopes proximally over a distal end of the proximal outer shaft.

19. The method of claim 18, wherein the first actuatable handle is positioned proximal to the second stationary handle.

20. The method of claim 18, wherein the first actuatable handle is positioned distal to the second stationary handle.
